(19) United States
(12) Patent Application Publication
(10) Pub. No.: US 2009/0264982 A1
(43) Pub. Date: Oct. 22, 2009

(54) Stent with auxiliary treatment structure

(76) Inventors: Arthur A. Krause, Winnetka, CA (US); Walter K. Lim, Santa Fe, CA (US)

Correspondence Address: Dennis H. Lambert, 7000 View Park Drive, Burke, VA, 22015, US

(21) Appl. No.: 12/456,891

(22) Filed: Jun. 24, 2009

Related U.S. Application Data
(62) Division of application No. 11/252,182, filed on Oct. 17, 2005.
(60) Provisional application No. 60/619,233, filed on Oct. 15, 2004.
(60) Provisional application No. 60/701,897, filed on Jul. 22, 2005.

(51) Int. Cl. A61F 2/06

(52) U.S. Cl. 623/1.15; 623/1.44

(57) ABSTRACT

A medical device for treatment of a stenosed body lumen, includes an open-ended cylindrical body carried on a distal end of a catheter for insertion of the device into the body lumen and placement at the stenosed site. The cylindrical body is movable between a collapsed position for insertion into the body lumen, and a radially expanded position pressed against the wall of the body lumen. In one embodiment the body sidewall is formed by a plurality of interconnected struts or elements defining openings therebetween, and at least one elongate ribbon is attached to an outer surface thereof for carrying a therapeutic agent. In another embodiment, the body is formed of interwoven elements defining a mesh-like structure, and the elements may comprise dissimilar materials, such as, e.g., copper and silver. In a further embodiment the body is formed of layers of different materials such as, e.g., copper, silver, and/or steel, laminated together. In a still further embodiment the device is designed for temporary placement of the catheter and body in a body lumen for treatment of a stenosed site, after which the catheter and body are withdrawn. In all forms the body may have an outwardly flared inlet end to reduce turbulence of fluid flowing through it, and/or a gel-like coating of a cholesterol-dissolving or blood clot dissolving agent may be placed on the device.

This application claims the benefit of U.S. provisional patent application Ser. Nos. 60/619,233, filed Oct. 15, 2004, and 60/701,897, filed Jul. 22, 2005.

### BACKGROUND OF THE INVENTION

1. Field of the Invention

The present invention relates to medical devices, and in particular to drug delivery stents, and to means for treatment of vascular disease at sites previously stented or previously unstented.

2. Description of the Related Art

Vascular disease leads to death or disability for tens of thousands of people each year in the United States alone. It is caused by progressive blockage, or stenosis, of the blood vessels that perfuse the heart and other major organs. More severe blockage of blood vessels in such individuals often leads to hypertension, ischemic injury, stroke, or myocardial infarction. Atherosclerotic lesions, which limit or obstruct coronary blood flow, are the major cause of ischemic heart disease.

The therapeutic alternatives available for treatment of stenosis include intervention (alone or in combination with therapeutic agents) to remove the blockage, replacement of the blocked segment with a new segment of artery, or the use of a catheter-mounted device such as a balloon catheter to dilate the artery. The dilation of an artery with a balloon catheter is called percutaneous transluminal angioplasty (PTA), while dilation of a coronary artery is called percutaneous transluminal coronary angioplasty (PTCA). PTCA is the predominant treatment for coronary vessel stenosis, and the increasing use of this procedure is attributable to its relatively high success rate and its minimal invasiveness compared with coronary bypass surgery. Both procedures are medical procedures whose purpose is to increase blood flow through an artery, and as used herein reference to one will be considered to generally apply to the other, unless otherwise indicated.

During angioplasty, a balloon catheter in a deflated state is inserted within a stenotic segment of a blood vessel and inflated and deflated one or more times to expand the vessel by compressing the built-up tissue or plaque in the vessel lumen to enlarge the opening and restore blood flow.

Angioplasty often permanently opens previously occluded blood vessels. However, a limitation associated with PTCA is the abrupt closure of the vessel that may occur immediately after the procedure, and restenosis, which occurs gradually following the procedure and refers to the re-narrowing of an artery after an initially successful angioplasty. Additionally, restenosis is a chronic problem in patients who have undergone saphenous vein bypass grafting. Post-angioplasty closure of the vessel, both immediately after PTCA (acute reocclusion) and in the long term (restenosis), is a major difficulty associated with PTCA.

The mechanism of acute occlusion appears to involve several factors and may result from vascular recoil with resultant closure of the artery and/or deposition of blood platelets and fibrin along the damaged length of the newly opened blood vessel.

The more gradual process of restenosis after PTCA is initiated by vascular injury resulting from balloon angioplasty, and 30% of patients with subtotal lesions and 50% of patients with chronic total lesions will go on to restenosis after angioplasty. Various processes, including thrombosis (clotting within a blood vessel), inflammation, growth factor and cytokine release, cell proliferation, cell migration and extracellular matrix synthesis each contribute to the restenotic process. While the exact mechanism of restenosis is not completely understood, the general aspects of the restenosis process have been identified. In the normal arterial wall, smooth muscle cells proliferate at a low rate, approximately less than 0.1 percent per day. Smooth muscle cells (SMC) in the vessel walls exist in a contractile phenotype characterized by eighty to ninety percent of the cell cytoplasmic volume occupied with the contractile apparatus. Endoplasmic reticulum, Golgi, and free ribosomes are few and are located in the perinuclear region. Extracellular matrix surrounds the smooth muscle cells and is rich in heparin-like glycosylaminoglycans which are believed to be responsible for maintaining smooth muscle cells in the contractile phenotypic state. The process of PTCA is believed to injure resident arterial smooth muscle cells (SMC). In response to this injury, adhering platelets, infiltrating macrophages, leukocytes, or the smooth muscle cells (SMC) themselves release cell-derived growth factors. Many other potential reasons are also being investigated.

Daughter cells migrate to the intimal layer of arterial smooth muscle and continue to proliferate and secrete significant amounts of extracellular matrix proteins. Proliferation, migration and extracellular matrix synthesis continue until the damaged endothelial layer is repaired at which time proliferation slows within the intima, usually within seven to fourteen days post-injury. The newly formed tissue is called neointima. The further vascular narrowing that occurs over the next three to six months is due primarily to negative or constrictive remodeling.

Simultaneous with local proliferation and migration, inflammatory cells adhere to the site of vascular injury. Within three to seven days post-injury, inflammatory cells have migrated to the deeper layers of the vessel wall. Inflammatory cells may persist at the site of vascular injury for at least thirty days. Inflammatory cells therefore may contribute to both the acute and chronic phases of restenosis.

Because 30-50% of patients undergoing PTCA will experience restenosis, the success of PTCA is clearly limited as a therapeutic approach to coronary artery disease. Because SMC proliferation and migration are intimately involved with the pathophysiological response to arterial injury, prevention of SMC proliferation and migration represents a target for pharmacological intervention in the prevention of restenosis.

In order to prevent restenosis and vessel collapse, stents of various configurations have been used to hold the lumen of a blood vessel open following angioplasty. Balloon angioplasty and associated implantation of a stent or stents compress the built-up tissue or plaque in a vessel lumen to enlarge the opening and restore blood flow. There is a multiplicity of different stents that may be utilized following percutaneous transluminal angioplasty. Examples are disclosed in U.S. Pat. Nos. 5,766,710, 6,254,632, 6,379,382 and 6,613,084, and in published US applications 2002/0062147, 2003/0065346, 2003/0105512, 2003/0125800, 2003/0181973, 2003/0225450 and 2004/0127977.

Most stents are compressible for insertion through small cavities, and are delivered to the desired implantation site percutaneously via a catheter or similar transluminal device. Once at the treatment site, the compressed stent is expanded to fit within or expand the lumen of the passageway. Stents are typically either self-expanding or are expanded by inflating a balloon that is positioned inside the compressed stent at the end of the catheter. Intravascular stents are often deployed after coronary angioplasty procedures to reduce complications, such as the collapse of arterial lining, associated with the procedure.

However, stents do not entirely reduce the occurrence of thrombotic abrupt closure due to clotting; stents with rough surfaces exposed to blood flow may actually increase thrombosis, and restenosis may still occur because tissue may grow through and around the lattice of the stent.

Thus, in addition to providing physical support to passageways, stents are also used to carry therapeutic substances for local delivery of the substances to the damaged vasculature. For example, anticoagulants, antiplatelets, and cytostatic agents are substances commonly delivered from stents and are used to prevent thrombosis of the coronary lumen, to inhibit development of restenosis, and to reduce post-angioplasty proliferation of the vascular tissue, respectively. The therapeutic substances are typically either impregnated into the stent or carried in a polymer that coats the stent. The therapeutic substances are released from the stent or polymer once it has been implanted in the vessel.

Numerous agents have been examined for presumed anti-proliferative actions in restenosis, including those identified in U.S. Pat. No. 6,379,382, the disclosure of which is incorporated herein. Some of the agents that have been shown to successfully reduce restenosis include: heparin and heparin fragments, colchicine, taxol, angiotensin converting enzyme (ACE) inhibitors, angiopeptin, and cyclosporin A.

The local delivery of drug/drug combinations from a stent is advantageous because it prevents vessel recoil and remodeling through the scaffolding action of the stent and the prevention of multiple components of neointimal hyperplasia or restenosis as well as a reduction in inflammation and thrombosis. This local administration of drugs, agents or compounds to stented coronary arteries may also have additional therapeutic benefit. For example, higher tissue concentrations of the drugs, agents or compounds may be achieved utilizing local delivery, rather than systemic administration.

In addition, reduced systemic toxicity may be achieved utilizing local delivery rather than systemic administration while maintaining higher tissue concentrations. Also in utilizing local delivery from a stent rather than systemic administration, a single procedure may suffice with better patient compliance. An additional benefit of combination drug, agent, and/or compound therapy may be to reduce the dose of each of the therapeutic drugs, agents or compounds, thereby limiting their toxicity, while still achieving a reduction in restenosis, inflammation and thrombosis. Local stent-based therapy is therefore a means of improving the therapeutic ratio (efficacy/toxicity) of anti-restenosis, anti-inflammatory, anti-thrombotic drugs, agents or compounds.

Coating of metal stents with a drug or beneficial agent generally requires the use of a polymer substrate to bond the agent to the stent, or stents with holes or depressions formed in them for storing the agent. Multiple drugs can be delivered by placing different drugs in different holes or depressions, or in different layers, but the holes or depressions tend to weaken the structure of the stent, and layering requires the drug carried by the underlying layer to pass through the top layer, or for the top layer to first dissolve or erode away.

Moreover, when reocclusion or restenosis occurs at a previously stented site, conventional practice involves the implantation of a further stent at that site, but normally only one such additional stent can be implanted. After that, if reocclusion or restenosis occurs it is generally necessary to perform bypass surgery.

Accordingly, it would be advantageous to provide a stent having means for simultaneous delivery of multiple drugs or beneficial agents to a traumatized site in a vessel lumen while avoiding the problems associated with the prior art. It would also be advantageous to provide a stent having an auxiliary structure attached to the stent for delivering different pharmacologic agents and/or providing other benefits. Further, it would be advantageous to provide a means for localized treatment of vascular disease without the need for implanting a stent, or for “repair” of previously stented sites without the need for implanting a second stent at the previously stented site.

### SUMMARY OF THE INVENTION

The device of the present invention has means for delivery of a therapeutic agent or agents to a stenosed site in a body lumen. The device includes an open-ended cylindrical body carried on a distal end of a catheter for insertion into the body lumen and placement at the stenosed site. The cylindrical body is movable between a collapsed position for insertion into the body lumen, and a radially expanded position pressed against the wall of the body lumen. In one embodiment, the device comprises a stent for permanent implantation, and the body sidewall is formed by a plurality of interconnected struts or elements defining openings therebetween, and at least one elongate ribbon is attached to an outer surface thereof for carrying a therapeutic agent. In another stent embodiment, the body is formed of interwoven elements defining a mesh-like structure, and the elements may comprise dissimilar materials, such as, e.g., copper and silver. In a further stent embodiment the body is formed of layers of different materials such as, e.g., copper, silver, and/or steel, laminated together. In a still further embodiment the device is designed for temporary placement of the catheter and body in a body lumen for treatment of a stenosed site, after which the catheter and body are withdrawn. In all forms the body may have an outwardly flared inlet end to reduce turbulence of fluid flowing through it, and/or a gel-like coating of a cholesterol-dissolving or blood clot dissolving agent may be placed on the device.

Those forms employing one or more ribbons, or an interwoven or a laminated structure, enable multiple drugs or beneficial agents to be delivered to a stenosed site without weakening the stent structure or necessarily layering drugs on the stent. In that form employing one or more ribbons, they are attached to the stent body in a manner to permit expansion of the stent, with one or more desirable beneficial agents impregnated in or placed on the ribbons for simultaneous release, whether over the same time interval or different time intervals.

The use of these separate treatment structures for delivering a desired medication avoids the problems associated with prior art devices, and also affords different and additional treatment options, as discussed more fully below.

The ribbons themselves in the first form of the invention, or the different layers of materials laminated together in the second form, or the different interwoven elements in the interwoven form, can be made of a material, such as copper, silver, steel, zinc, chrome, carbon, gold, brass, tantalum, titanium, platinum, sulfur compounds, and/or alloys or compositions thereof, that produce beneficial biological results when placed in a body lumen. Copper ions, for example, catalyze the breakdown of blood chemicals called nitrosothiols, thereby releasing nitric oxide, and nitric oxide prevents clot formation on implants.

In one embodiment according to the first form of the invention, the ribbon or ribbons can comprise laminated layers of different metals and/or metal alloys, e.g., copper/stainless steel/zinc, or combinations of other materials and alloys to achieve a desired result. Alternatively, multiple ribbons made of different materials can be applied to the surface of a stent so as to extend in generally side-by-side relationship to one another rather than laminated in different layers.

In another embodiment, the ribbons of the invention may be made of woven strands of copper, silver, steel, or other materials to expose multiple metals or other materials to the area of the stent implant.

The ribbons are fastened at least at one end to the near or proximal end of the stent, and in one embodiment extend generally straight along the length of the stent on its outer surface. In another embodiment, the ribbon may have a zig-zag shape, and in a further embodiment the ribbon or ribbons may be wrapped or wound around the stent in a spiral pattern. If made of relatively loosely woven strands, for example, the ribbons may be stretchable and in that event could be secured to the stent at both ends.

The surface of the ribbon or portions of it can be roughened or given a texture, or the ribbon can have holes formed in it to facilitate binding of a drug or beneficial agent to the ribbon without requiring the use of a polymer substrate or formation of holes or depressions in the stent itself. Discrete patches or nodules of different agents can be placed in different locations on the ribbons (either on the roughened or textured areas, or in different holes, or on opposite surfaces of the ribbon or ribbons.

The ribbon can be used in combination with a bare metal stent or a drug-eluting stent, and may have a material on it that dissolves plaque (a biofilm). Naturally occurring compounds such as Lecithin, Allicin (a raw garlic extract) and/or onion extracts, and HDL (high density lipoprotein) are examples of compounds that are known to dissolve or liquefy plaque. After the plaque liquefying/dissolving agents do their work, the blood will carry the dissolved plaque to be removed by the kidneys. Ideally, thus removing the obstruction from the artery.

A medication or different medications can be applied intermittently to spaced areas of the ribbon or ribbons, with the material of the ribbon exposed between the spaced areas. The exposed areas of the ribbon thus can provide or produce additional biological or pharmacological effects. For example, if the ribbon is made of copper or silver it can impede or prevent restenosis through the production of, e.g., copper ions that catalyze the breakdown of blood chemicals called nitrosothiols, thereby releasing nitric oxide, and nitric oxide prevents clot formation on implants. If one uses copper ions as a preventative for stenosis and restenosis, then it is not necessary to put drugs or medications on the stent for this same purpose.

The ribbons may be made dissolvable, in the manner of dissolvable sutures, for timed release of pharmacological agents embedded in the ribbon, or for other desired purposes.

The different layers of material in the laminated structure of the second form of the invention can be selected to obtain a desired result based on the known properties of the materials, e.g., a central layer or lamination of stainless steel can be sandwiched between inner and outer layers of copper and/or zinc or other relatively malleable material to provide strength to the structure.

Various therapeutic substances can be provided on the stent. For example, anticoagulants, antiplatelets, and cytostatic agents are substances commonly delivered from stents and are used to prevent thrombosis of the coronary lumen, to inhibit development of restenosis, and to reduce post-angioplasty proliferation of the vascular tissue, respectively. Compounds such as Lecithin, Allicin (a raw garlic extract) and/or onion extracts, and HDL, are examples of naturally occurring compounds that can be used. Other examples include those identified in U.S. Pat. No. 6,379,382, the disclosure of which is incorporated herein, and heparin and heparin fragments, colchicine, taxol, angiotensin converting enzyme (ACE) inhibitors, angiopeptin, and cyclosporin A.

The therapeutic substances listed are exemplary only, and are not intended to be limiting on the present invention.

Further, the means for localized treatment of vascular disease without the need for implanting a stent, or for “repair” of previously stented sites without the need for implanting a second stent at the previously stented site, comprises a catheter with a device on its distal end for temporary placement at the diseased site and delivery for a limited time of a therapeutic agent or treatment that dissolves plaque or otherwise treats the diseased site as desired or necessary. The device preferably has means that permits blood to continue flowing past the site while treatment is being performed.

In all forms of the invention the device may have a flared inlet end to reduce turbulence of fluid flowing through it, and/or a gel-like substance selected for its ability to dissolve plaque or blood clots, for example, may be coated on the device.

### BRIEF DESCRIPTION OF THE DRAWINGS

The foregoing, as well as other objects and advantages of the invention, will become apparent from the following detailed description when taken in conjunction with the accompanying drawings, wherein like reference characters designate like parts throughout the several views, and wherein:

FIG. 1 is an enlarged fragmentary view in side elevation of a first embodiment of stent having ribbons attached to it in accordance with the invention, wherein the ribbons extend longitudinally of the stent in generally straight, parallel relationship to one another.

FIG. 1A is a fragmentary enlarged view of a portion of the device of FIG. 1, showing the flared inlet end.

FIG. 2 is an enlarged fragmentary view in side elevation of a second embodiment of stent having ribbons attached to it in accordance with the invention, wherein the ribbons are wound around the stent in a spiral pattern, extending longitudinally of the stent in generally parallel relationship to one another.

FIG. 3 is an enlarged fragmentary view in side elevation of a third embodiment of stent having ribbons attached to it in accordance with the invention, wherein the ribbons are applied to the stent in a zig-zag pattern, extending longitudinally of the stent in generally parallel relationship to one another.

FIG. 4 is a greatly enlarged fragmentary plan view of a portion of a ribbon having intermittent, spaced roughened or textured areas on it for holding a drug or other beneficial agent.

FIG. 5 is a greatly enlarged fragmentary plan view of a portion of a ribbon having intermittent, spaced openings or holes formed through it for holding a drug or other beneficial agent.

FIG. 6 is a taken along line 6-6 in FIG. 5.

FIG. 7 is a longitudinal sectional view similar to FIG. 6, of a ribbon having intermittent, spaced recesses or depressions formed in it for holding a drug or other beneficial agent.

FIG. 8 is a greatly enlarged fragmentary plan view of a portion of a ribbon comprising woven strands according to an embodiment of the invention.

FIG. 9 is a side view of the woven ribbon of FIG. 8.

FIG. 9A is a fragmentary enlarged view of a portion of the device of FIG. 9, showing the flared inlet end.

FIGS. 10-12 are greatly enlarged fragmentary side views in elevation of typical prior art stents with which the present invention may be used.

FIG. 13 is an exploded perspective view depicting several sheets of material in position to be laminated together in a multi-layered structure or substrate for use in forming a stent of generally tubular configuration.

FIG. 14 is a transverse sectional view of several layers of material laminated together to form a sheet used in forming a tubular stent.

FIG. 15 is a perspective view of an example of a stent that can be made using multiple layers of material laminated together.

FIG. 15A is a fragmentary enlarged view of a portion of the device of FIG. 15, showing the flared inlet end.

FIG. 16 is a somewhat schematic longitudinal side view of a device for therapeutic treatment of previously stented sites or previously unstented sites.

FIG. 17 is a somewhat schematic enlarged fragmentary longitudinal sectional view of the device of FIG. 16, shown in place in a body lumen.

FIG. 18 is a transverse sectional view taken along line 18-18 in FIG. 17.

FIG. 19 is a fragmentary, somewhat schematic longitudinal sectional view depicting a generic stent or stent-like body having a gel-like coating on its inner and outer surfaces.

### DETAILED DESCRIPTION OF PREFERRED EMBODIMENTS

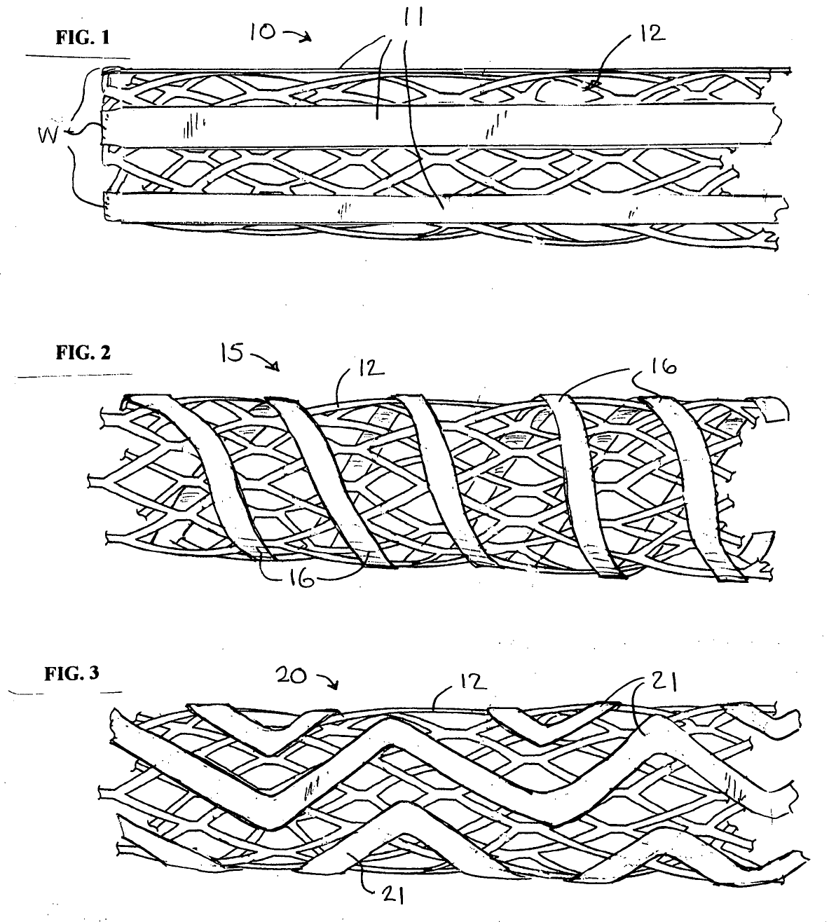

A first embodiment of a stent with auxiliary treatment structure according to the invention is shown generally at 10 in FIG. 1. In this embodiment, a plurality of relatively wide bands or ribbons 11 are attached at least at one end to one end of the stent 12, and extend generally straight and parallel to one another longitudinally of the stent. The stent may be of any suitable construction, and in the example shown is of the type depicted in FIG. 10.

In order to permit expansion of the stent, the ribbons preferably are attached to the stent at only one end. In some stent constructions, the ribbons may be attached to both ends of the stent, and when the stent is expanded radially, it can shrink axially to accommodate expansion, even with the ribbons attached to both ends of the stent. Attachment of the ribbons can be by welding or other means known in the art, as represented at W in FIG. 1. Although not shown, it should be understood that the following embodiments could be similarly secured.

A second embodiment is shown at 15 in FIG. 2, wherein the ribbons 16 are wound around the stent 12 in a spiral pattern. As in the previous form, the ribbons can be attached at only one end or at both ends, depending upon the structure of the stent and the ability of the stent to undergo radial expansion with the ribbons attached.

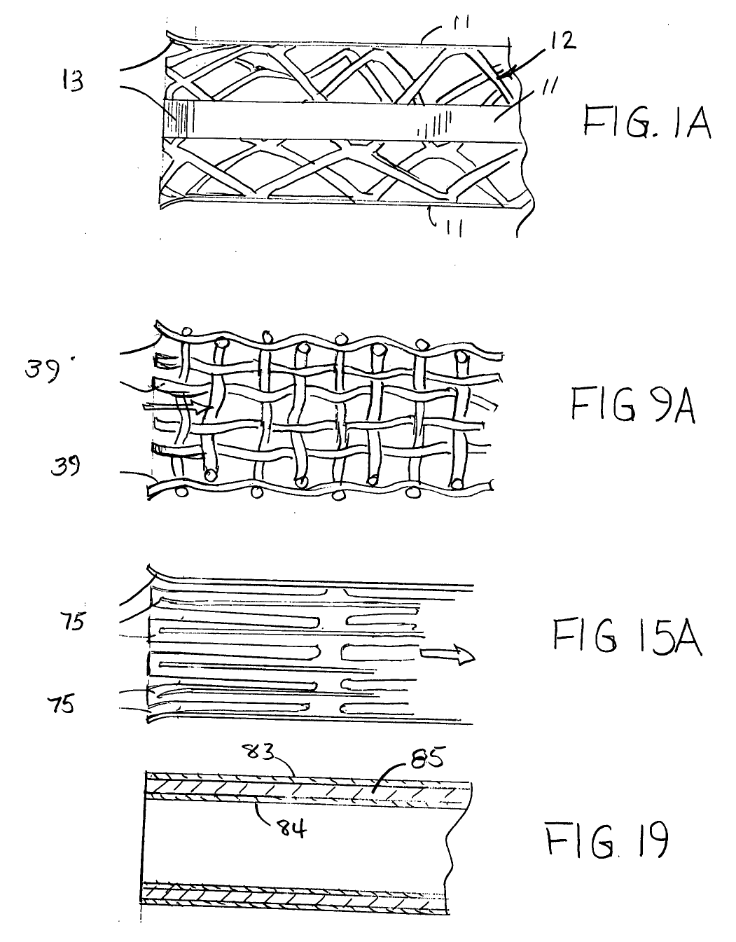

A third embodiment is shown at 20 in FIG. 3, wherein the ribbons 21 are applied to the stent 12 in a zig-zag pattern. As in the previous form, the ribbons can be attached at only one end or at both ends, depending upon the structure of the stent and the ability of the stent to undergo radial expansion with the ribbons attached.

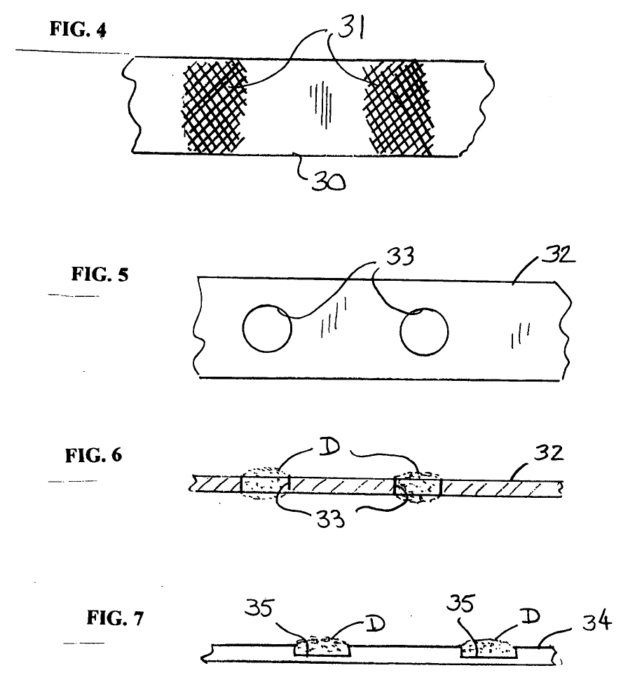

FIG. 4 depicts a ribbon 30 having roughened or textured areas 31 on its surface to provide a surface for enhanced mechanical bonding of a drug or other beneficial agent to the surface of the ribbon.

FIGS. 5 and 6 depict a ribbon 32 having openings or holes 33 formed through it to provide a means for applying a drug or other beneficial agent D to the ribbon.

FIG. 7 depicts a ribbon 34 having recesses or depressions 35 formed in the surface to provide a means for applying a drug or other beneficial agent D to the ribbon.

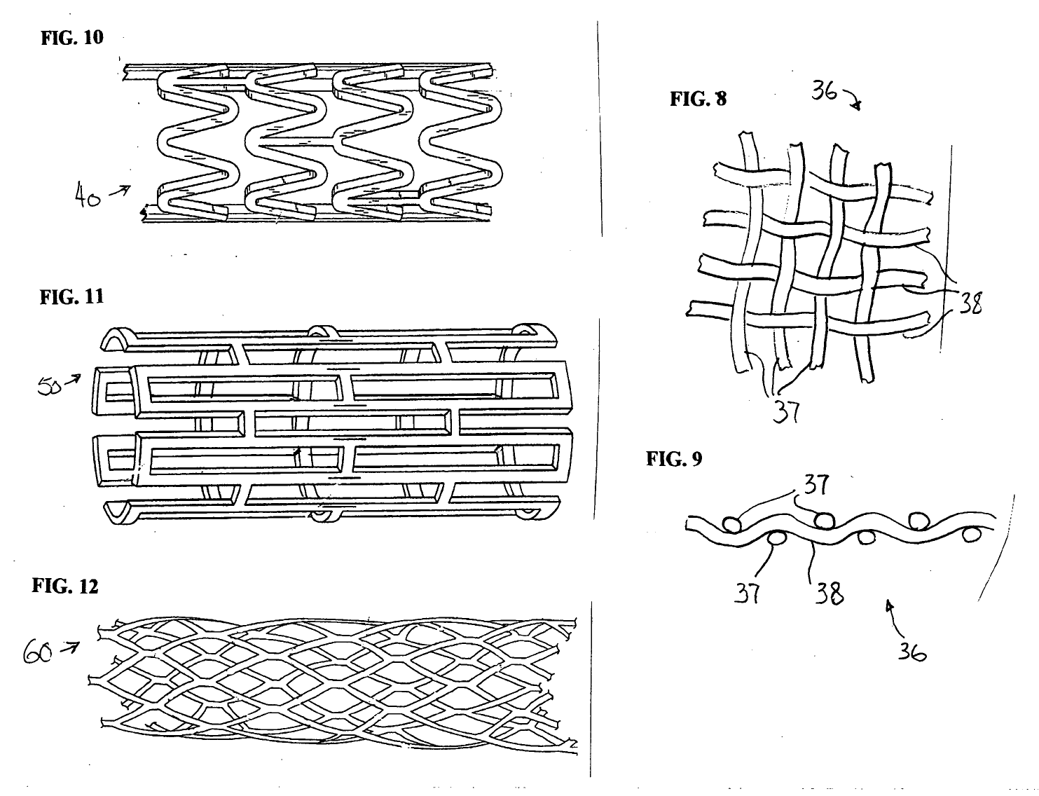

FIGS. 8 and 9 depict a section 36 of a stent body or a ribbon made of interwoven strands of material 37 and 38. The strands 37 and 38 could comprise one or more different materials, such as copper, sliver, and the like, and when the stent or ribbon 36 is implanted at a stenosed site, the different materials are exposed to the tissue. The strands can have the same or different medications coated on or impregnated in them.

The stents 40, 50 and 60, shown in FIGS. 10-12, respectively, are exemplary of different stent structures that can be used in practicing the present invention, but other stent designs could be used.

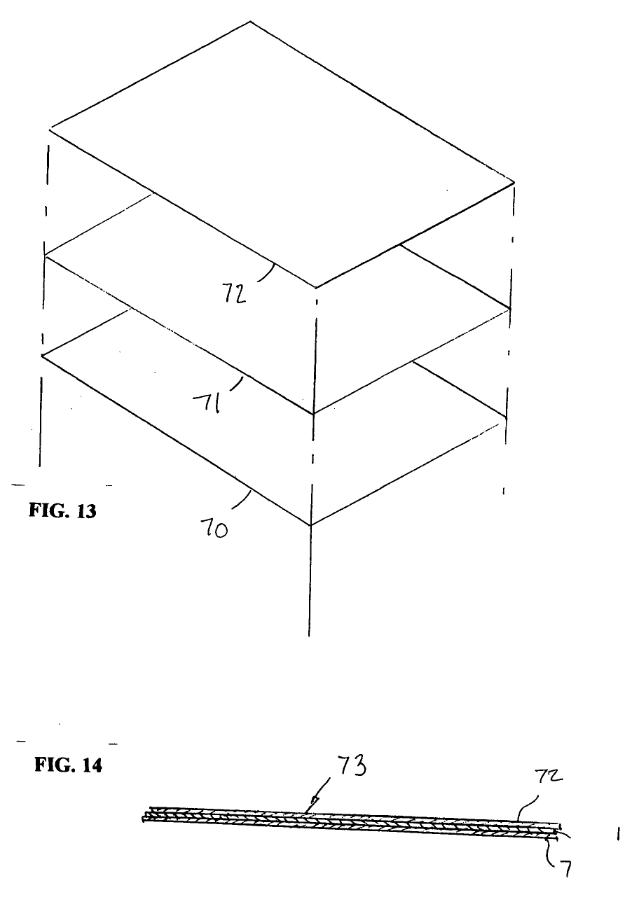

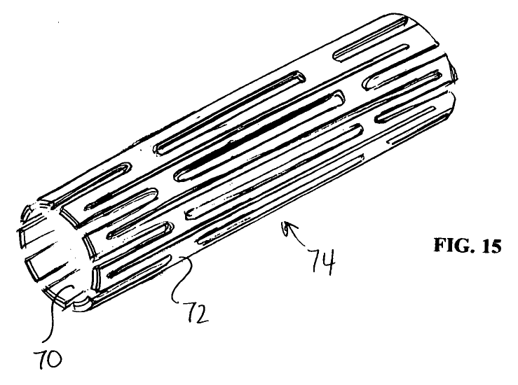

FIGS. 13-15 depict how multiple layers of one or more materials 70, 71 and 72 may be laminated together to form a sheet 73 that can then be formed into a tubular structure and cut with a laser or other known process to produce a stent such as shown at 74, for example, with layer 70 exposed to the blood on the interior of the stent, and layer 72 exposed to the vessel wall. Lamination of the layers may be accomplished in accordance with conventional processes, e.g., they may be cold-pressed together under sufficient pressure to fuse the layers together, or the layers may be welded together, etc. Similarly, forming of the tubular structure and cutting it into a desired design can be accomplished using known processes and techniques. One or more of the layers can be produced by ion deposition, or by powder coating, or other processes for coating one material onto another.

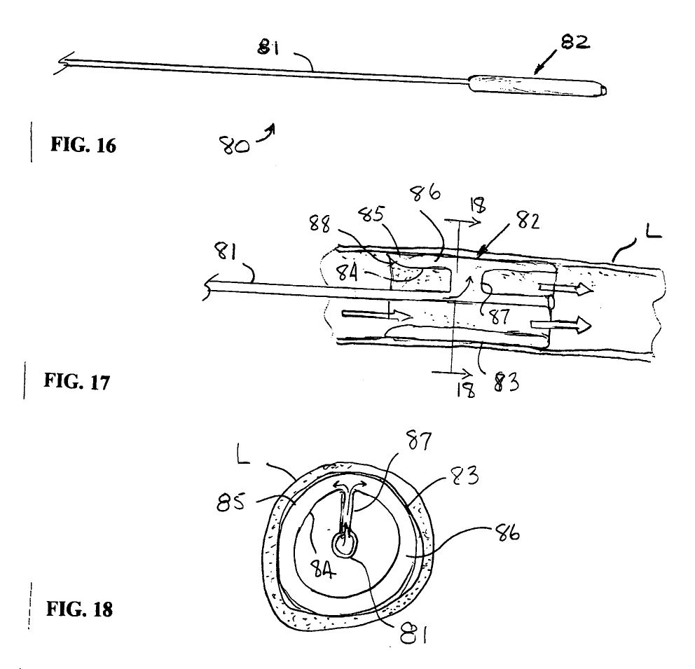

FIGS. 16, 17 and 18 depict a device 80 for temporary insertion into a body lumen L to treat a diseased or occluded site in the lumen. The device includes a catheter 81 similar to the type used conventionally to implant a stent, with an expandable structure 82 on its distal end. The expandable structure includes an outer, expandable, open-ended, double-walled cylinder 83 designed for carrying on its outer surface a therapeutic agent selected for treatment of the diseased site, such as dissolving plaque at the site, or performing other treatment as desired or necessary. The device can be positioned at a site for appropriate treatment of the site, in lieu of implanting a stent, or it can be positioned in a previously stented site to treat restenosis at the site, thereby avoiding the necessity of implanting a second stent.

In the embodiment shown, the double-walled cylinder 83 comprises an inflatable structure of stretchable elastomeric material, having an inner cylindrical wall 84 and an outer cylindrical wall 85, defining an annular space 86 therebetween. The space is connected to an inflation tube (not shown) in the catheter so that air or other fluid can be pumped into the space to inflate the cylinder. The cylinder remains collapsed on the distal end of the catheter 81, as depicted in FIG. 16, until the cylinder is positioned at the desired site, whereupon it can be inflated and expanded to the configuration shown in FIGS. 17 and 18, with the drug-carrying outer surface of the cylinder pressed against the lumen wall. As seen best in FIG. 18, the space 86 is connected to the inflation tube in the catheter via one or more radially extending members 87. The member 87 preferably is narrow in a direction transverse to the direction of blood flow, whereby it minimally interferes with flow. It can be one or more simple cylindrical tubes (not shown), or an axially elongate structure as shown in FIG. 17, or a connection similar to that shown in FIG. 17, but extending throughout the length of the wall 84 can be used (not shown). Any of these arrangements provide a flow passage through the center of the structure 82 for continuous flow of blood while the device is in place. Moreover, the single radial member shown induces minimal turbulence in blood flowing through the device, but is sufficient to inflate it.

The device is left in place a predetermined time, e.g., 5 to 30 minutes, for appropriate treatment of the site, and is then collapsed and withdrawn from the lumen. For example, the device could be temporarily positioned at a diseased site to dissolve plaque or perform other treatment without the need for implantation of a stent. Or if restenosis occurs in a previously stented site, the device could be placed temporarily at the site to treat the restenosis without the need for implanting a second stent at the site.

Although the device 82 has been shown and described as inflatable, it should be understood that other expandable and retractable means could be employed, so long as space is left through the device for continued flow of blood while the device is in place. For instance, a mechanism similar to that used on an umbrella could be employed, with suitable cables or wires extended through the catheter for manipulating linkages to expand and contract the device.

All forms of the invention could have an outwardly flared inlet end to reduce turbulence of fluid flowing through the device, as depicted at 13 in FIG. 1A, 39 in FIG. 9A, and 75 in FIG. 15A. Further, a coating of a gel-like substance selected for its ability to dissolve plaque or blood clots, for example, could be coated on one or both the outer and inner surfaces of all forms of the invention, as depicted at 83, 84 and 85 in FIG. 19, wherein 83 represents the coating on an outer surface, 84 represents the coating on an inner surface, and 85 represents a stent body for permanent implantation or the body of a device for temporary placement.

While particular embodiments of the invention have been illustrated and described in detail herein, it should be understood that various changes and modifications may be made in the invention without departing from the spirit and intent of the invention as defined by the appended claims.

What is claimed is:

1-17. (canceled)

18. A stent for implantation into a treatment site in a body lumen, comprising:
an elongate, open-ended tubular stent body having a sidewall of interconnected lattice elements or struts defining a plurality of openings through the sidewall, said sidewall being movable from a collapsed position on an end of a catheter for insertion into a body lumen, to a radially expanded position engaged against an inner surface of the body lumen, wherein:
said stent body comprises a lamination of different metals.

19. A stent as claimed in claim 18, wherein:
said stent body comprises a lamination of two different metals.

20. A stent as claimed in claim 19, wherein:
one of said metals comprises copper and the other comprises stainless steel.

21. A stent as claimed in claim 20, wherein:
the stainless steel lamination comprises an inner tubular support structure, and the copper lamination comprises an outer layer on the stainless steel support structure.

22. A stent as claimed in claim 18, wherein:
said stent body comprises a lamination of three different metals.

23. A stent body as claimed in claim 22, wherein:
there is an inner lamination of silver, an intermediate lamination of stainless steel, and an outer lamination of copper.

24. A stent for implantation into a treatment site in a body lumen, comprising:
an elongate, open-ended tubular stent body comprising a plurality of interwoven elements defining a plurality of openings through the sidewall, said sidewall being movable from a collapsed position on an end of a catheter for insertion into a body lumen, to a radially expanded position engaged against an inner surface of the body lumen, wherein:
said interwoven elements include elements of at least two different materials.

25. A stent as claimed in claim 24, wherein:
said interwoven elements include first elements of silver and second elements of copper.

26-32. (canceled)

33. A medical device for insertion into a stenosed site in a body lumen to treat and remove the stenosis, comprising:
an elongate tubular body having an open inlet end, an open outlet end, and a sidewall, said body being movable from a collapsed position on an end of a catheter for insertion into a body lumen, to a radially expanded position engaged against an inner surface of the body lumen, wherein said open inlet end is outwardly flared to provide smooth entry for fluid flowing through said body, thereby reducing turbulence in said fluid.

34. (canceled)
